(19) United States
(12) Patent Application Publication
(10) Pub. No.: US 2025/0146975 A1
(43) Pub. Date: May 8, 2025

(54) DETECTION DEVICE

(71) Applicant: TAIYO YUDEN CO., LTD., Tokyo (JP)

(72) Inventor: Tomoaki KANETO, Tokyo (JP)

(73) Assignee: TAIYO YUDEN CO., LTD., Tokyo (JP)

(21) Appl. No.: 19/018,429

(22) Filed: Jan. 13, 2025

Related U.S. Application Data
(63) Continuation of application No. PCT/JP2023/021878, filed on Jun. 13, 2023.

(30) Foreign Application Priority Data
Jul. 29, 2022 (JP) 2022-122167

(51) Int. Cl. G01N 29/02; G01N 33/00

(52) U.S. Cl. CPC G01N 29/02; G01N 33/0027; G01N 2291/014; G01N 2291/021

(57) ABSTRACT

A detection device includes a vibrator having a sensitive membrane, a heater configured to heat the sensitive membrane, a detector configured to detect a detection value related to a resonance frequency of the vibrator, a control unit configured to cause the heater to start heating the sensitive membrane, acquire a first detection value detected by the detector in a state where the sensitive membrane is heated, and cause the heater to stop heating the sensitive membrane based on the first detection value and a first reference value, and an arithmetic unit configured to acquire a second detection value related to a gas to be measured detected by the detector after heating of the sensitive membrane is stopped, and calculate determination information about the gas based on the second detection value.

### CROSS-REFERENCE TO RELATED APPLICATION

This application is a continuation application of PCT/JP2023/021878 filed on Jun. 13, 2023, which claims priority to Japanese Patent Application No. 2022-122167 filed on Jul. 29, 2022, the entire contents of which are incorporated herein by reference.

### FIELD

A certain aspect of the present disclosure relates to a detection device.

### BACKGROUND

As a detection device for detecting information on a gas such as the type of a substance in the gas or odor, there is a detection device in which a sensitive membrane is provided on a vibrator. When a specific substance in the gas is adsorbed on the sensitive membrane, the sensitive membrane becomes heavier, and the oscillation frequency of the vibrator decreases. It is known to perform a refresh operation or a cleaning operation for desorbing the substance adsorbed on the sensitive membrane as disclosed in, for example, Japanese Patent Application Laid-Open Nos. 2019-146675 and 2010-117184 (Patent Documents 1 and 2, respectively). It is known that the oscillation frequency after the refresh operation is set as a reference frequency and the information on the gas is calculated based on a difference between the oscillation frequency and the reference frequency as disclosed in, for example, Patent Document 1.

### SUMMARY

If molecules of water or the like have already been adsorbed on the sensitive membrane, molecules of a substance to be detected are less likely to be adsorbed on the sensitive membrane. This reduces the detection sensitivity of the information on the gas. In one aspect of the present disclosure, there is provided a detection device including: a vibrator having a sensitive membrane; a heater configured to heat the sensitive membrane; a detector configured to detect a detection value related to a resonance frequency of the vibrator; a control unit configured to cause the heater to start heating the sensitive membrane, acquire a first detection value detected by the detector in a state where the sensitive membrane is heated, and cause the heater to stop heating the sensitive membrane based on the first detection value and a first reference value; and an arithmetic unit configured to acquire a second detection value related to a gas to be measured detected by the detector after heating of the sensitive membrane is stopped, and calculate determination information about the gas based on the second detection value.

### BRIEF DESCRIPTION OF THE DRAWINGS

FIG. 1 is a schematic view of a vibrator in a first embodiment;

FIG. 2 is a block diagram of a detection device in the first embodiment;

FIG. 3 is a flowchart illustrating a process executed by a processing unit in the first embodiment;

FIG. 4 is a flowchart illustrating the procedure of Experiment 1;

FIG. 5A and FIG. 5B are graphs presenting the sensitivity |f2-fref2| with respect to periods T1 to T7 in Experiment 1;

FIG. 6 is a graph presenting the amount of change in oscillation frequency, f1′-fref1′, with respect to periods T1 to T7 in Experiment 1;

FIG. 7A and FIG. 7B are graphs presenting the sensitivity |f2-fref2| with respect to the amount of change in oscillation frequency, f1′-fref1′, in sensitive membranes A and B in Experiment 1, respectively;

FIG. 8A and FIG. 8B are graphs presenting the sensitivity |f2-fref2| with respect to the amount of change in oscillation frequency, f1′-fref1′, in sensitive membranes C and D in Experiment 1;

FIG. 9 is a flowchart illustrating a process executed by the processing unit in a first variation of the first embodiment;

FIG. 10 is a flowchart illustrating a process executed by the processing unit in a second variation of the first embodiment; and

FIG. 11 is a block diagram of a detection device in a second embodiment.

### DETAILED DESCRIPTION

Hereinafter, embodiments will be described with reference to the drawings.

### First Embodiment

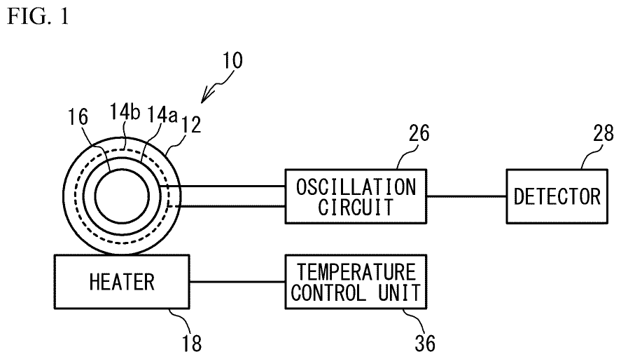

A quartz crystal microbalance (QCM) using a quartz vibrator as a vibrator will be described as an example. FIG. 1 is a schematic view of a vibrator in a first embodiment. As illustrated in FIG. 1, a vibrator 10 includes a quartz plate 12 and electrodes 14a and 14b that sandwich the quartz plate 12 therebetween. A sensitive membrane 16 is provided on the electrode 14a. The vibrator 10 is provided on a heater 18. The heater 18 may be spaced from the vibrator 10. The heater 18 may be provided in another location as long as the heater 18 can heat the sensitive membrane 16 to a predetermined temperature. The electrodes 14a and 14b are electrically connected to an oscillation circuit 26. The oscillation circuit 26 oscillates at an oscillation frequency related to the resonance frequency of the vibrator 10. The resonance frequency of the vibrator 10 can be defined as either the resonance frequency (fr) at the resonance point or the anti-resonance frequency (fa) at the anti-resonance point of the vibrator 10, or any frequency between them. The oscillation circuit 26 is considered to oscillate at a frequency near the resonance frequency. The increased mass of the vibrator 10 due to the adhesion of a specific substance to the sensitive membrane 16 lowers the resonance frequency, which in turn lowers the oscillation frequency. A detector 28 detects the oscillation frequency as a detection value related to the resonance frequency of the vibrator 10. The heater 18 is controlled by a temperature control unit 36.

The quartz plate 12 is a monocrystalline quartz, for example, an AT-cut quartz substrate. The electrodes 14a and 14b are metal layers mainly composed of gold or copper, for example. The heater 18 is, for example, a heater, and is a conductor wire such as a platinum wire or a nickel-chromium wire. The sensitive membrane 16 can be heated by applying a voltage to both ends of the conductor wire of the heater 18.

The material of the sensitive membrane 16 is, for example, a polymer material, a porous material, or an organic metal compound. Examples of the polymer material include cellulose, a fluorine-based polymer, polyethyleneimine, an ester-based polymer, an acryl-based polymer, polystyrene, polybutadiene, and a cycloolefin polymer, and the polymer material has a functional group to which a specific substance easily binds. The porous material is, for example, zeolite or metal organic framework (MOF) such as UiO-66 or ZIF-8. The organic metal compound is, for example, a metallophthalocyanine or a metalloporphyrin. The metal of the organic metal compound is, for example, copper, nickel, cobalt, or zinc.

When a polymer material is used as the sensitive membrane 16, the decomposition temperature is preferably 120° C. or higher so that the sensitive membrane 16 is not decomposed by heating. The glass transition temperature of the sensitive membrane 16 is preferably 100° C. or higher. When cellulose is used as the sensitive membrane 16, the decomposition temperature is generally around 150° C., but when cellulose having high heat resistance is used, the decomposition temperature is about 300° C. In the case of a fluorine-based polymer, for example, the decomposition temperature of poly vinylidene difluoride (PVDF) is 150° C. to 170° C., and the decomposition temperature of polytetrafluoroethylene (PTFE) is about 330° C. In the case of polyethyleneimine, the decomposition temperature is about 270° C. In the case of polystyrene, the decomposition temperature is 330° C. to 370° C. In the case of polybutadiene, the decomposition temperature is 430° C. The sensitive membrane 16 is selected in consideration of the decomposition temperature and the glass transition temperature of the material to be used.

When moisture or other molecules in the gas are adsorbed on the sensitive membrane 16, the mass of the sensitive membrane 16 increases. This lowers the resonance frequency of the vibrator 10, resulting in a lower oscillation frequency. The temperature control unit 36 causes the heater 18 to heat the sensitive membrane 16, so that moisture or other molecules adsorbed on the sensitive membrane 16 are desorbed.

As the vibrator 10, instead of the quartz vibrator, a vibrator using a piezoelectric layer such as a surface acoustic wave (SAW) resonator or a bulk acoustic wave (BAW) resonator such as a film bulk acoustic resonator (FBAR) or a solidly mounted resonator (SMR) can be used. The substance in the gas to be detected is, for example, an organic compound such as ethanol, acetone, or toluene, or an inorganic substance such as ammonia, nitrogen oxide, ozone, or chlorine.

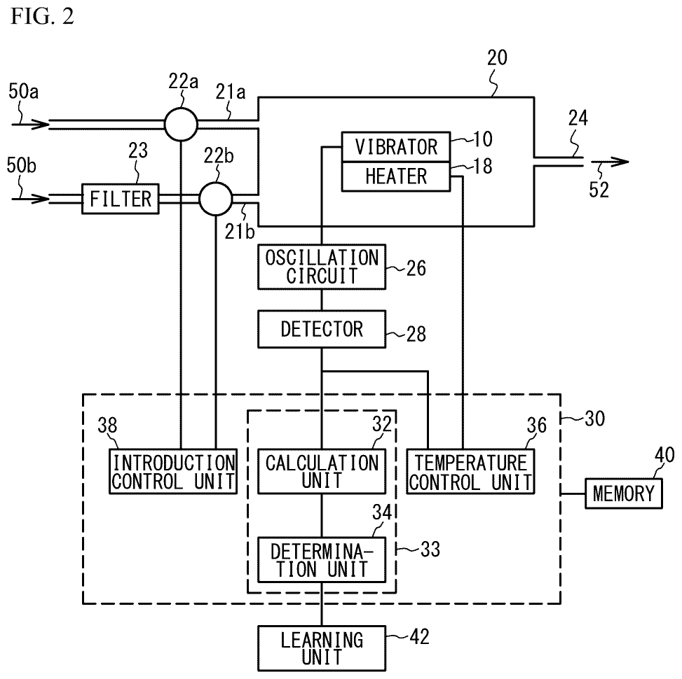

FIG. 2 is a block diagram of the detection device in the first embodiment. As illustrated in FIG. 2, the vibrator 10 and the heater 18 are provided in a chamber 20. Gases 50a and 50b are introduced into the chamber 20 from introduction paths 21a and 21b, and a gas 52 is discharged from a discharge path 24. A pump 22a is provided in the introduction path 21a, and by driving the pump 22a, the gas 50a outside of the chamber 20 is introduced into the chamber 20. A filter 23 and a pump 22b are provided in the introduction path 21b. By driving the pump 22b, the gas 50b outside of the chamber 20 is introduced into the chamber 20 through the filter 23. The filter 23 is, for example, active carbon, zeolite, silica gel, and/or molecular sieves, and removes moisture, specific molecules and the like in the gas 50b. Therefore, by driving the pump 22b, dry and clean air is introduced into the chamber 20. The gas introduced from the introduction path 21a into the chamber 20 is a gas to be measured containing a detection target such as a specific substance in the gas or odor. For example, if there is a generation source that generates odor in the environment surrounding the chamber 20, the outside air contains a specific substance, odor, or the like. Therefore, the outside air can be used as the gas to be measured by taking the outside air into the chamber 20. The gas introduced from the introduction path 21b into the chamber 20 is a reference gas, for example, dry and clean air. The reference gas may be any gas as long as it contains almost no detection target, and for example, a cylinder filled with an inert gas or the like may be connected to the introduction path 21a without providing the filter 23, and the inert gas or the like may be introduced into the chamber 20.

The detector 28 detects an oscillation frequency related to the resonance frequency of the vibrator 10 as a detection value. A processing unit 30 is, for example, a processor. The processing unit 30 cooperates with software to function as a calculation unit 32, a determination unit 34, the temperature control unit 36, and an introduction control unit 38. An arithmetic unit 33 functions as the calculation unit 32 and the determination unit 34. At least a part of the processing unit 30 may be hardware such as a dedicated circuit.

The calculation unit 32 calculates sensitivity and the like based on the detection value such as the oscillation frequency detected by the detector 28. The determination unit 34 calculates determination information on the gas based on the sensitivity or the like calculated by the calculation unit 32. The determination information on the gas will be described later. A memory 40 is, for example, a volatile memory or a nonvolatile memory, and stores the detection value such as the oscillation frequency detected by the detector 28. A learning unit 42 stores a machine learning model used for determination by the determination unit 34. The learning unit 42 relearns the machine learning model based on the calculation result of the calculation unit 32. One or both of the determination unit 34 and the learning unit 42 may be provided in the detection device configured by hardware or may be provided on a server connected through a network such as a cloud.

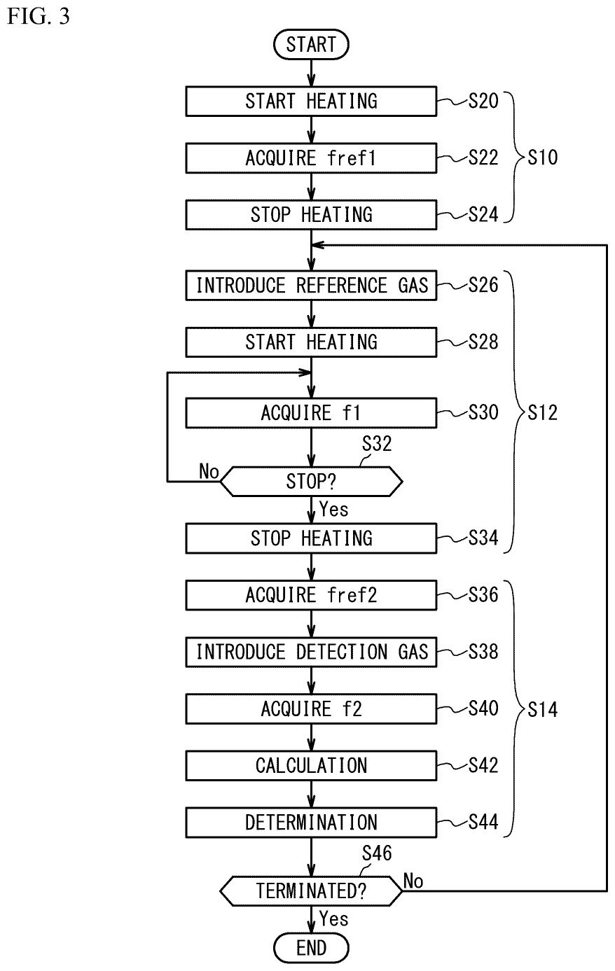

FIG. 3 is a flowchart illustrating a process executed by the processing unit in the first embodiment. As illustrated in FIG. 3, the process executed by the processing unit 30 is roughly separated into steps S10, S12, and S14. Step S10 is the process of acquiring an initial first reference value fref1. Step S12 is the process of heating the sensitive membrane 16 in order to desorb moisture and other molecules adsorbed on the sensitive membrane 16. Step S14 is the process of calculating determination information on the gas.

First, as step S10, the temperature control unit 36 causes the heater 18 to start heating the sensitive membrane 16 (step S20). The temperature of the sensitive membrane 16 before heating and the temperature of the sensitive membrane 16 after a certain time has elapsed after stopping heating are substantially the temperature of the surrounding environment, for example, room temperature. The room temperature in this case is any temperature in a range of 0° C. to 40° C. The heating temperature of the sensitive membrane 16 is, for example, 100° C. to 300° C. or 150° C. to 250° C., and is 230° C. as an example. The heating time is, for example, 1 to 10 minutes, and is 5 minutes as an example. The heating temperature and heating time of the sensitive membrane 16 are set to a temperature and time at which moisture and other molecules adsorbed on the sensitive membrane 16 can be sufficiently desorbed. The detector 28 acquires the oscillation frequency output from the oscillation circuit 26 as the first reference value fref1 (step S22). The processing unit 30 stores the first reference value fref1 in the memory 40. The temperature control unit 36 causes the heater 18 to stop heating the sensitive membrane 16 (step S24). Through the above steps, step S10 is completed. Step S10 is performed, for example, when the vibrator 10 is used for the first time. Step S10 is performed by introducing a reference gas into the chamber 20, for example. Through the above steps, step S10 is completed.

Thereafter, as step S12, the introduction control unit 38 drives the pump 22b to introduce the reference gas from the introduction path 21b into the chamber 20 (step S26). The temperature control unit 36 causes the heater 18 to start heating the sensitive membrane 16 (step S28). The heating temperature of the sensitive membrane 16 is the same as the heating temperature of the sensitive membrane 16 in step S20. The detector 28 acquires the oscillation frequency output from the oscillation circuit 26 as a first detection value f1 (step S30). The temperature control unit 36 acquires the first reference value fref1 from the memory 40, and determines whether to stop heating based on the first detection value f1 and the first reference value fref1 (step S32). The temperature control unit 36 determines Yes when the difference between the first detection value f1 and the first reference value fref1, |f1-fref1|, is within a certain range, and determines No when the difference is outside the certain range, for example. When the determination is No, the process returns to step S30. The sensitive membrane 16 is heated until the difference |f1-fref1| becomes within the certain range. When the determination is Yes in step S32, the temperature control unit 36 causes the heater 18 to stop heating the sensitive membrane 16 (step S34). Through the above processes, step S12 is completed.

Then, as step S14, the detector 28 acquires the oscillation frequency output by the oscillation circuit 26 as a second reference value fref2 (step S36). The processing unit 30 stores the second reference value fref2 in the memory 40. After stopping the pump 22b, the introduction control unit 38 drives the pump 22a to introduce the detection gas from the introduction path 21a into the chamber 20 (step S38). The detector 28 acquires the oscillation frequency output from the oscillation circuit 26 as a second detection value f2 (step S40). The period from the introduction of the detection gas to the detection of the second detection value f2 is a period until the second detection value f2 is stabilized, and is, for example, 5 minutes. The calculation unit 32 acquires the second reference value fref2 from the memory 40 and calculates the sensitivity based on the second reference value fref2 and the second detection value f2 (step S42). The sensitivity is, for example, f2-fref2. The determination unit 34 calculates the determination information about the gas based on the calculated sensitivity and the like (step S44).

The determination information about the gas is, for example, the type or concentration of a substance in the gas, the type of odor of the gas, the intensity of the odor, or the like. The type of substance in the gas is, for example, an ethanol molecule or an acetone molecule. The type of odor of the gas is determined by a complex combination of molecules such as ethanol molecules and acetone molecules and the amount (ratio) of each molecule, and is, for example, a cigarette odor or an aging odor. The intensity of the odor of the gas is an index indicating how strong the odor is, for example, how strong the odor such as a cigarette odor or an aging odor is. Through the above processes, step S14 is completed. The processing unit 30 determines whether to terminate the process (step S46). For example, when steps S12 and S14 are repeated a desired number of times (one or more times), the determination becomes Yes. When the determination is No, the process returns to step S12.

### Experiment 1

The following experiments were conducted. The vibrator 10 used in the experiment was made of quartz having dimensions of 1.25 mm×1.7 mm×0.0506 mm, and has a resonance frequency of approximately 32 MHz at 25° C. The vibrators 10 using the following four materials as the sensitive membrane 16, respectively, were fabricated.
Sensitive membrane A: Cycloolefin polymer having a fluorine-based functional group
Sensitive membrane B: Cycloolefin polymer having carboxylic acid
Sensitive membrane C: Polyimide having a hexafluoroisopropyl group
Sensitive membrane D: Polyacetylene having a silyl group

The following gases were used as detection gases.
Sensitive membranes A to C: Air containing ethanol at a concentration of 50 ppm
Sensitive membrane D: Air containing toluene at a concentration of 50 ppm

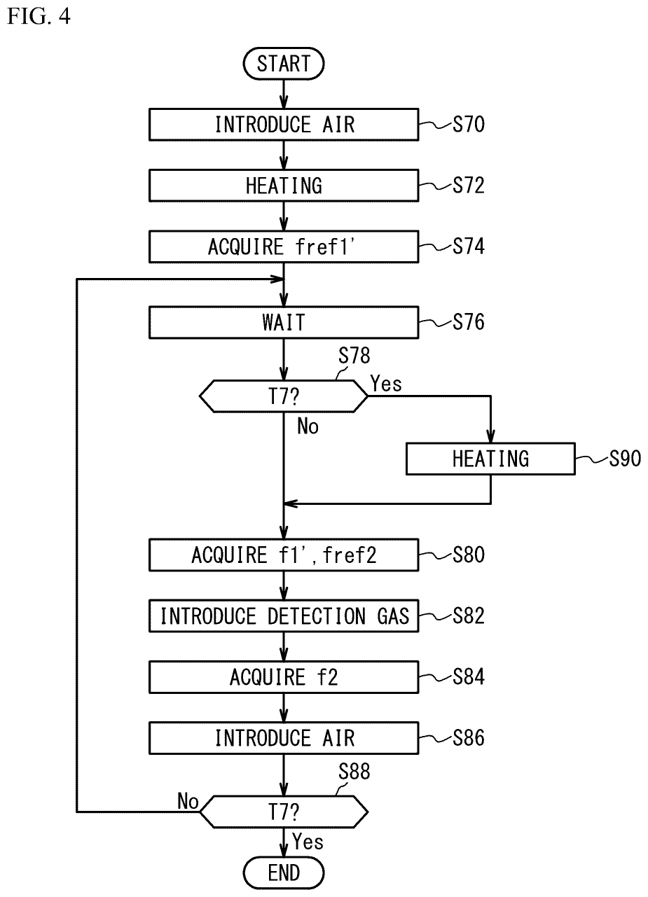

FIG. 4 is a flowchart illustrating the procedure of Experiment 1. As illustrated in FIG. 4, air is introduced into the chamber 20 (step S70). The sensitive membrane 16 is heated at 230° C. for 5 minutes (step S72). After the sensitive membrane 16 is cooled to room temperature, the oscillation frequency is acquired as the first reference value fref1′ (step S74). In Experiment 1, the first reference value fref1′ is 31.85 MHz. Then, the process waits for a predetermined period Ti (i is an integer from 1 to 7) from step S74 (step S76). It is determined whether the period is T7 (step S78). When the determination is No, the oscillation frequency is acquired as the first detection value f1′ and the second reference value fref2 (step S80). The detection gas is introduced into the chamber 20 (step S82). The oscillation frequency is acquired as the second detection value f2 (step S84). Air is introduced into the chamber 20 (step S86). It is determined whether the period is T7 (step S88). When the determination is No, i is incremented, and the process returns to step S76 and waits for the period T2. Then, steps S78 to S88 are repeated. When the determination is Yes in step S78, the sensitive membrane 16 is heated at 230° C. for 5 minutes (step S90). Then, steps S80 to S86 are performed. When the determination is Yes in step S88, the process is finished.

In FIG. 4, the periods T1 to T7 in step S74 were set as follows.
Period T1: 0 days
Period T2: 3 days
Period T3: 14 days
Period T4: 4 weeks
Period T5: 6 weeks
Period T6: 8 weeks
Period T7: 10 weeks

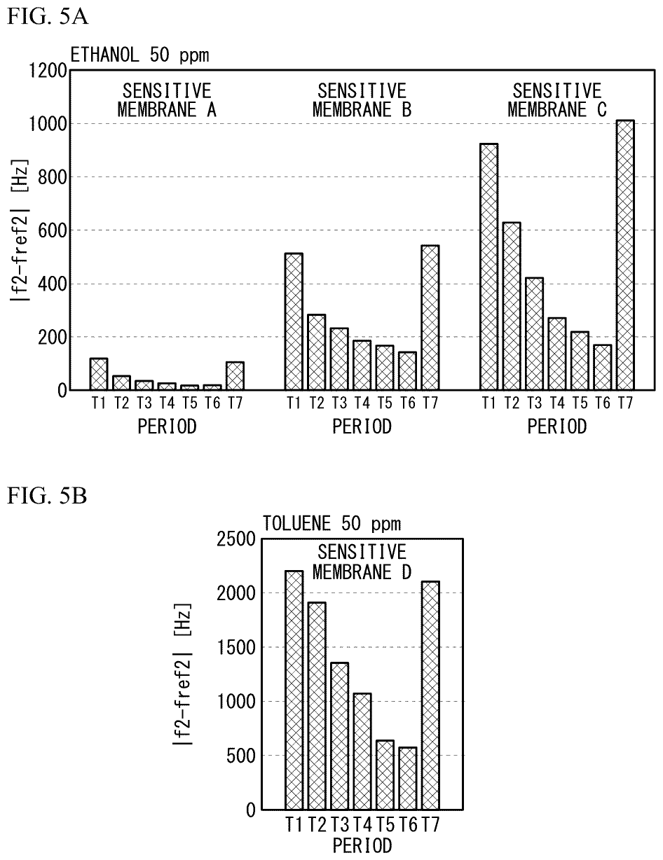

FIG. 5A and FIG. 5B are graphs presenting the sensitivity |f2-fref2| with respect to the periods T1 to T7 in Experiment 1. The horizontal axis represents the periods T1 to T7, and the vertical axis represents the absolute value of the difference between the second detection value f2 and the second reference value fref2 acquired in steps S84 and S80 of FIG. 4, respectively, in each of the periods T7 to T1. FIG. 5A presents the results obtained when air containing ethanol at a concentration of 50 ppm was used as the detection gas for the sensitive membranes A, B, and C, and FIG. 5B presents the results obtained when air containing toluene at a concentration of 50 ppm was used as the detection gas for the sensitive membrane D.

As presented in FIG. 5A and FIG. 5B, the sensitivity |f2-fref2| in the period T1 varies depending on the sensitive membranes A to D. The sensitivity for the period T1 is the sensitivity immediately after heating the sensitive membrane 16. As the period goes from T2 to T6, the period after heating the sensitive membrane 16 becomes longer. As the periods T2 to T6 become longer, the sensitivity decreases. This is considered to be because moisture and other molecules are adsorbed on the sensitive membrane 16 when time has elapsed after the sensitive membrane 16 is heated, and thus molecules of the substance to be detected in the detection gas are less likely to be adsorbed on the sensitive membrane 16. When the sensitive membrane 16 is heated again after the period T7, the sensitivity returns to the sensitivity after the period T1. This is considered to be because the moisture and other molecules adsorbed on the sensitive membrane 16 are desorbed from the sensitive membrane 16 by heating the sensitive membrane 16 again, and thereby molecules of the substance to be detected in the detection gas are easily adsorbed to the sensitive membrane 16.

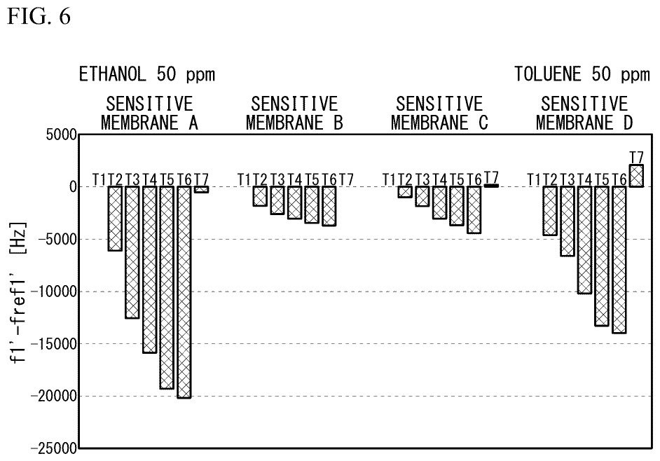

FIG. 6 is a graph presenting the amount of change in oscillation frequency, f1′-fref1′, with respect to the periods T1 to T7 in Experiment 1. The horizontal axis indicates the periods T1 to T7, and the vertical axis indicates the amount of change f1′-fref1′, which is the difference between the first reference value fref1′ acquired in step S74 in FIG. 4 and the first detection value f1′ acquired in step S80, and is the amount of change in oscillation frequency from the first reference value fref1′ as the initial oscillation frequency immediately after heating the sensitive membrane 16.

As presented in FIG. 6, for the period T1, f1′-fref1′=0 in any of the sensitive membranes A to D. As the periods T2 to T6 become longer, the amount of change f1′-fref1′ increases in the negative direction. This is considered to be because moisture and other molecules are adsorbed on the sensitive membrane 16 when time elapses after the sensitive membrane 16 is heated, and thus the mass of the sensitive membrane 16 increases. When the sensitive membrane 16 is heated again after the period T7, the amount of change f1′-fref1′ returns to the amount of change f1′-fref1′ after the period T1. This is considered to be because the moisture and other molecules adsorbed on the sensitive membrane 16 are desorbed from the sensitive membrane 16 by heating the sensitive membrane 16 again, and thus the mass of the sensitive membrane 16 returns to the original level.

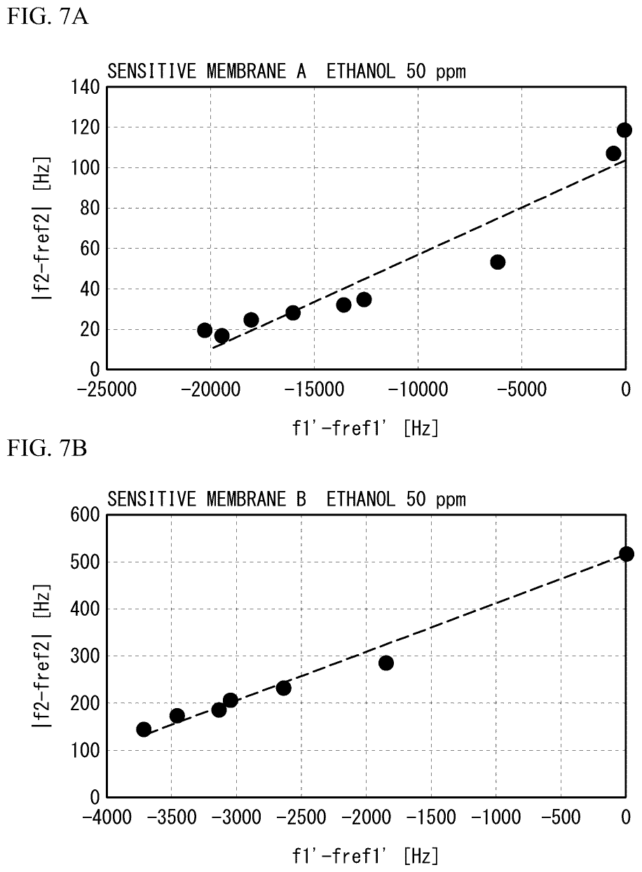

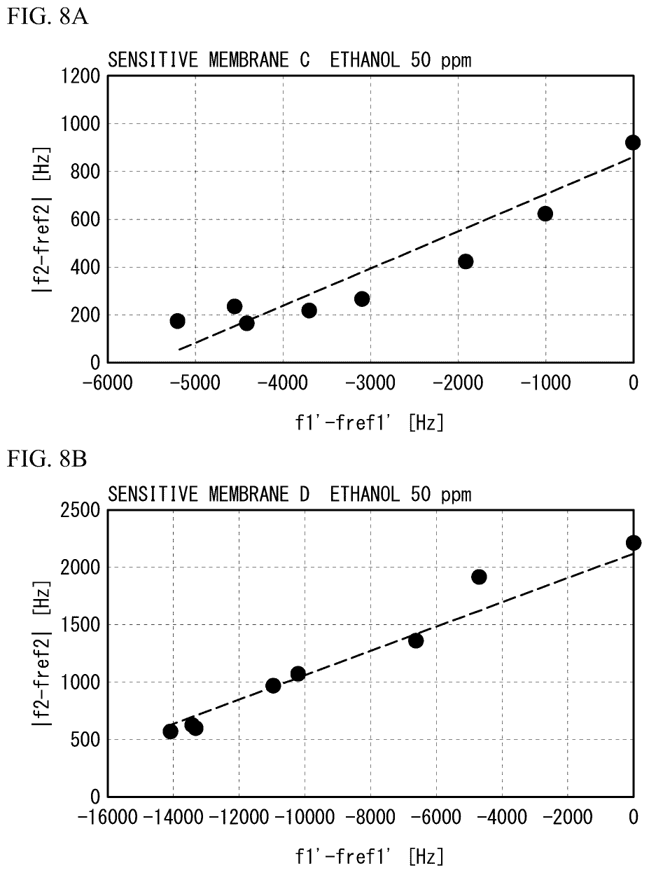

FIG. 7A to FIG. 8B are graphs presenting the sensitivity |f2-fref2| with respect to the amount of change in oscillation frequency f1′-fref1′ in the sensitive membranes A to D of Experiment 1, respectively. Dots represent measurement points, and a broken line is an approximate straight line calculated by using the least squares method.

As presented in FIG. 7A to FIG. 8B, in any of the sensitive membranes A to D, when the amount of change in oscillation frequency f1′-fref1′ increases in the negative direction, the sensitivity |f2-fref2| decreases. This is because when the amount of change in oscillation frequency f1′-fref1′ is large in the negative direction, moisture or other molecules are already adsorbed on the sensitive membrane 16, and molecules of the substance to be detected are less likely to be adsorbed. Therefore, it is considered that the sensitivity is lowered. In any of the cases of FIG. 7A to FIG. 8B, the coefficient of determination R² when the approximate straight line is calculated using the least squares method is 0.9 or greater, and the correlativity between f1′-fref1′ and |f2-fref2| is very high.

As in Experiment 1, when moisture and other molecules are adsorbed on the sensitive membrane 16 after the sensitive membrane 16 is heated, the oscillation frequency is lowered. When moisture and other molecules are desorbed from the sensitive membrane 16 by heating the sensitive membrane 16, the oscillation frequency returns to the original level. However, if the desorption of moisture and other molecules from the sensitive membrane 16 is not sufficient, the oscillation frequency will not return to the original level. When a specific substance in the gas is detected in this state, the sensitivity is reduced as presented in FIG. 7A to FIG. 8B. Further, if the amount of moisture and other molecules desorbed from the sensitive membrane 16 is not constant, the sensitivity of detection of the specific substance varies, and the reproducibility is lowered. Although it is possible to sufficiently desorb moisture and other molecules from the sensitive membrane 16 by sufficiently heating the sensitive membrane 16, the sensitive membrane 16 deteriorates when the heating temperature is high. As the heating temperature is lowered, the heating time increases and the detection time thereby increases.

Therefore, according to the first embodiment, as described in step S28 of FIG. 3, the temperature control unit 36 (control unit) causes the heater 18 to start heating the sensitive membrane 16. As described in step S30, the temperature control unit 36 acquires the first detection value f1 detected by the detector 28 while the sensitive membrane 16 is heated. As described in steps S32 and S34, the temperature control unit 36 causes the heater 18 to stop heating the sensitive membrane 16 based on the first detection value f1 and the first reference value fref1. As described in step S40, the calculation unit 32 acquires the second detection value f2 (detection value related to the gas to be measured) detected by the detector 28 while the sensitive membrane 16 is exposed to the gas to be detected after the heating of the sensitive membrane 16 is stopped. As described in steps S42 and S44, the determination unit 34 calculates the determination information about the gas based on the second detection value f2. In this manner, the heater 18 is caused to stop heating the sensitive membrane 16 based on the first detection value f1 and the first reference value fref1. This allows the determination unit 34 to calculate the determination information about the gas based on the second detection value f2 after the moisture and other molecules adsorbed on the sensitive membrane 16 are desorbed to a certain extent. Therefore, it is possible to reduce the variation in sensitivity due to the adsorption of moisture and other molecules on the sensitive membrane 16.

In Experiment 1, the first reference value fref1′ and the first detection value f1′ are oscillation frequencies when the temperature of the sensitive membrane 16 is at substantially room temperature, whereas in the first embodiment, the first reference value fref1 and the first detection value f1 are oscillation frequencies while the sensitive membrane 16 is heated. In consideration of the change in the resonance frequency of the vibrator 10 due to the temperature, it is considered that the first reference value fref1 and the first detection value f1 during heating behave similarly to the first reference value fref1′ and the first detection value f1′ at room temperature.

The temperature control unit 36 causes the heater 18 to stop heating the sensitive membrane 16 when the difference between the first detection value f1 and the first reference value fref1, |f1-fref1| becomes equal to or less than a threshold value. When fref1>f1, the difference between f1 and fref1 is fref1-f1. This allows moisture and other molecules adsorbed on the sensitive membrane 16 to be desorbed to a certain extent. Therefore, it is possible to further reduce the variation in sensitivity due to the adsorption of moisture and other molecules on the sensitive membrane 16.

As described in step S22, the first reference value fref1 is an initial value of the detection value related to the resonance frequency detected by the detector 28 in the state where the sensitive membrane 16 is heated. Thus, in step S12, the sensitive membrane 16 is heated until the first detection value f1 returns to the first reference value fref1. Thus, the amount of moisture and other molecules adsorbed on the sensitive membrane 16 can be brought into the initial state, and therefore, the sensitivity can be improved to the initial level.

In step S32, the reference value when stopping the heating of the sensitive membrane 16 is smaller than the initial value (fref1) acquired in step S22. For example, when the first detection value f1 in step S30 is, for example, 80% of the first reference value fref1, the temperature control unit 36 determines Yes in step S32. In this manner, heating of the sensitive membrane 16 is stopped before the first detection value f1 returns to the first reference value fref1. Thus, the heating temperature can be lowered, and the deterioration of the sensitive membrane 16 can be suppressed. In addition, the heating time can be shortened, and the detection time can be shortened.

In step S36, the calculation unit 32 acquires the second reference value fref2 (reference value related to the reference gas) detected by the detector 28 in the state where the sensitive membrane 16 is exposed to the reference gas after the heating of the sensitive membrane 16 is stopped. In step S44, the determination unit 34 calculates the determination information about the gas based on the second detection value f2 and the second reference value fref2. For example, in step S42, the calculation unit 32 calculates the sensitivity |f2-fref2| from the second detection value f2 and the second reference value fref2, and in step S44, the determination unit 34 calculates the determination information about the gas based on the sensitivity. This allows the determination information about the gas to be calculated with high accuracy.

### First Variation of First Embodiment

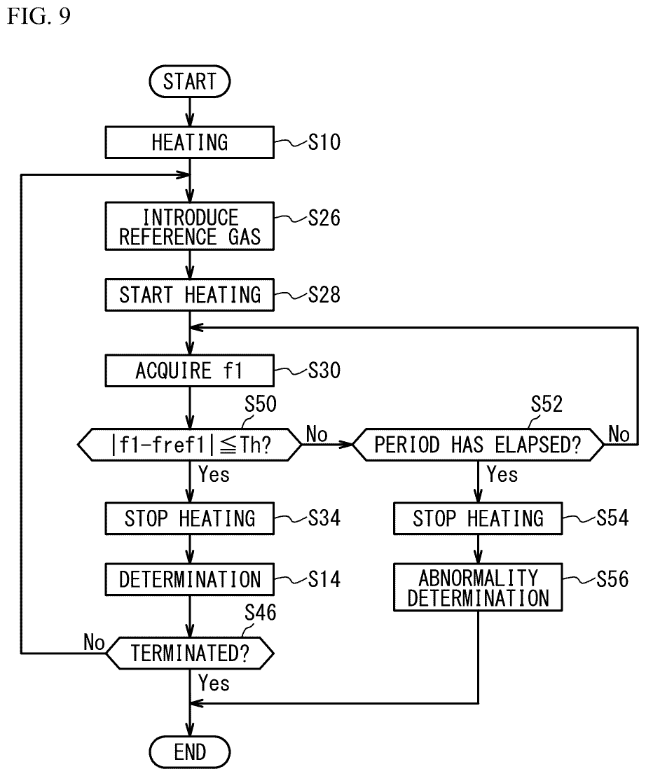

FIG. 9 is a flowchart illustrating a process executed by the processing unit in a first variation of the first embodiment. As illustrated in FIG. 9, in the first variation of the first embodiment, the processes executed by the processing unit 30 up to step S30 are the same as those of the first embodiment illustrated in FIG. 3. The temperature control unit 36 determines whether |f1-fref1|≤Th (step S50). When the determination is Yes, the temperature control unit 36 causes the heater 18 to stop heating the sensitive membrane 16 (step S34). Then, the processing unit 30 executes the processes in and after step S14 in FIG. 3.

When the determination is No in step S50, the temperature control unit 36 determines whether a predetermined period of time has elapsed from the start of heating in step S28 (step S52). When the determination is No, the process returns to step S30. When the determination is Yes, the temperature control unit 36 causes the heater 18 to stop heating the sensitive membrane 16 (step S54). The temperature control unit 36 determines that there is an abnormality (step S56). Then, the process is terminated. Other processes executed by the processing unit 30 are the same as those in FIG. 3 of the first embodiment, and the description thereof will be omitted.

When the molecules adsorbed on the sensitive membrane 16 adhere to it, the adhered molecules are not desorbed even if the sensitive membrane 16 is heated. Therefore, the first detection value f1 does not recover to the first reference value fref1. Therefore, the temperature control unit 36 determines that there is an abnormality when the difference |f1-fref1| does not become equal to or less than the threshold value Th even after a predetermined period of time has elapsed from the start of heating of the sensitive membrane 16. This allows the sensitive membrane 16 to be determined to have reached the end of its life, and the vibrator 10 having the sensitive membrane 16 can be replaced.

### Second Variation of First Embodiment

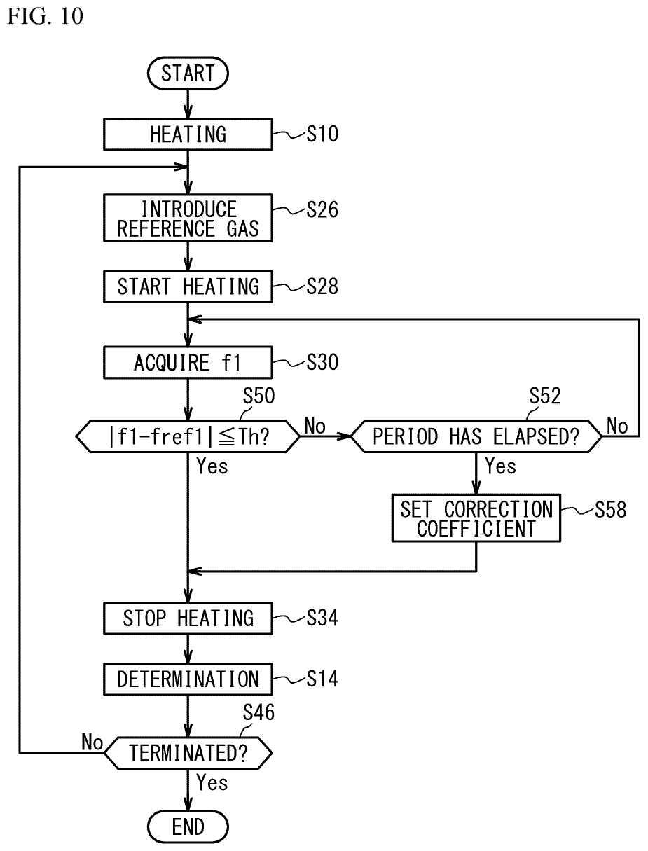

FIG. 10 is a flowchart illustrating processes executed by the processing unit in a second variation of the first embodiment. As illustrated in FIG. 10, in the second variation of the first embodiment, the processes executed by the processing unit 30 up to step S30 are the same as those of the first embodiment illustrated in FIG. 3. The temperature control unit 36 determines whether |f1-fref1|≤Th (step S50). When the determination is No, the temperature control unit 36 determines whether a predetermined period of time has elapsed from the start of heating in step S28 (step S52). When the determination is No, the process returns to step S30. When the determination is Yes, the temperature control unit 36 sets a correction coefficient Co (step S58). Then, the process proceeds to step S34. In step S42 (see FIG. 3) of step S14, the calculation unit 32 calculates the sensitivity using the correction coefficient Co. For example, the calculation unit 32 calculates |f2-fref2|×Co as the sensitivity. Other processes executed by the processing unit 30 are the same as those of FIG. 3 of the first embodiment, and the description thereof will be omitted.

The first detection value f1 does not recover to the first reference value fref1 when the difference |f1-fref1| does not become equal to or less than the threshold value Th even after a predetermined period of time has elapsed since the start of heating of the sensitive membrane 16. In this case, as illustrated in FIG. 7A to FIG. 8B, the sensitivity |f2-fref2| decreases. Therefore, the arithmetic unit 33 corrects the second detection value f2 based on the first detection value f1 and the first reference value fref1, and calculates the determination information about the gas based on the corrected second detection value f2. For example, in the case of the sensitive membrane A, when the first detection value f1 is smaller than the first reference value fref1 by 5000 Hz, as illustrated in FIG. 7A, the sensitivity |f2-fref2| is approximately 0.8 times the sensitivity |f2-fref2| when f1′-fref1′=0. Therefore, in step S58, the calculation unit 32 sets the correction coefficient Co to 1/0.8=1.25. In step S42 (see FIG. 3), the calculation unit 32 calculates |f2-fref2|×Co as the sensitivity. Thus, even when the sensitive membrane 16 is deteriorated, the determination information about the gas can be accurately calculated.

In the first variation of the first embodiment, the correction coefficient may be used as in the second variation. For example, when the threshold values of the first and second variations of the first embodiment are Th1 and Th2, respectively, Th1 is set to be larger than Th2. As a result, when the difference |f1-fref1| is larger than Th2 and equal to or smaller than Th1, the sensitivity is calculated using the correction coefficient as in steps S58 and S14 of FIG. 10, and the sensitive membrane 16 is used. When the difference |f1-fref1| is larger than Th2, it is determined that there is an abnormality as in step S56 of FIG. 9.

### Second Embodiment

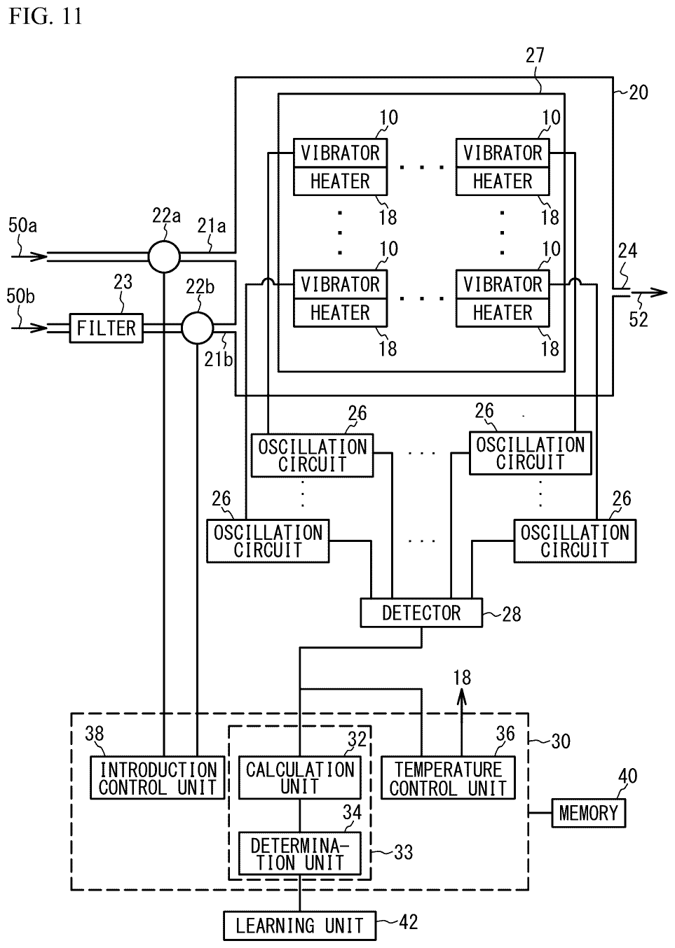

FIG. 11 is a block diagram of a detection device in a second embodiment. As illustrated in FIG. 11, in the second embodiment, a substrate 27 is provided in the chamber 20. A plurality of the vibrators 10 are provided on the substrate 27. The vibrators 10 are arranged in a matrix of 4×4, for example. The heater 18 for heating the sensitive membrane 16 is provided for each vibrator 10. The oscillation circuits 26 output signals with oscillation frequencies related to the resonance frequencies of the vibrators 10, respectively. The detector 28 detects the oscillation frequencies of the oscillation circuits 26 as detection values. The temperature control unit 36 causes the heaters 18 to heat the respective vibrators 10.

The calculation unit 32 acquires the first reference value fref1, the first detection value f1, the second reference value fref2, and the second detection value f2 for each of the vibrators 10, and calculates the sensitivity and the like for each of the vibrators 10. The determination unit 34 calculates determination information about the gas using the sensitivities and the like calculated for the respective vibrators 10 as feature amounts. Other configurations are the same as those of the first embodiment and the variations thereof.

In the second embodiment, the sensitive membrane 16 of at least one of the vibrators 10 is made of a material different from that of the sensitive membrane 16 of another vibrator 10. Thus, the behavior of the sensitivity or the like of each vibrator 10 varies depending on the type of substance in the gas to be detected. Therefore, the arithmetic unit 33 calculates the determination information about the gas based on the second detection values f2 corresponding to the respective vibrators 10. By using a large number of feature amounts, it is possible to calculate the determination information about the gas with higher accuracy.

One heater 18 may be provided in common for the vibrators 10. The temperature and time at which the adsorbed molecules and the like are desorbed differ depending on the type of the sensitive membrane 16. Even in such a case, the heating temperature and the heating time of the sensitive membranes 16 of the vibrators 10 are the same. Therefore, if the heating time is adjusted to the sensitive membrane 16 having the longest desorption time of moisture or the like, the heating time becomes too long for other sensitive membranes 16. On the other hand, if the heating temperature is set to the sensitive membrane 16 having the highest desorption temperature of moisture or the like, the heating temperature is too high for other sensitive membranes 16, and the sensitive membranes 16 deteriorate.

Therefore, in the second embodiment, the heater 18 is provided for each vibrator 10, and the temperature control unit 36 heats the sensitive membrane 16 of at least one vibrator 10 of the vibrators 10 to a temperature different from that of the sensitive membrane 16 of another vibrator 10. For example, the heating temperatures are made different between the vibrators 10 having the sensitive membranes 16 made of different materials. Thus, the heating temperature can be increased for the sensitive membrane 16 having a high temperature at which the specific substance is desorbed. The heating temperature for other sensitive membranes 16 can be lowered. Therefore, the heating time can be shortened. Further, the deterioration of the sensitive membrane 16 can be suppressed.

Although the embodiment of the present disclosure has been described in detail above, the present disclosure is not limited to the specific embodiment, and various modifications and changes can be made within the scope of the gist of the present disclosure described in the claims.

What is claimed is:

1. A detection device comprising:
a vibrator having a sensitive membrane;
a heater configured to heat the sensitive membrane;
a detector configured to detect a detection value related to a resonance frequency of the vibrator;
a control unit configured to cause the heater to start heating the sensitive membrane, acquire a first detection value detected by the detector in a state where the sensitive membrane is heated, and cause the heater to stop heating the sensitive membrane based on the first detection value and a first reference value; and
an arithmetic unit configured to acquire a second detection value related to a gas to be measured detected by the detector after heating of the sensitive membrane is stopped, and calculate determination information about the gas based on the second detection value.

2. The detection device according to claim 1, wherein the control unit causes the heater to stop heating the sensitive membrane when a difference between the first detection value and the first reference value becomes equal to or less than a threshold value.

3. The detection device according to claim 1, wherein the first reference value is an initial value of a detection value related to the resonance frequency detected by the detector in the state where the sensitive membrane is heated.

4. The detection device according to claim 1, wherein the first reference value is smaller than an initial value of a detection value related to the resonance frequency detected by the detector in the state where the sensitive membrane is heated.

5. The detection device according to claim 2, wherein the control unit determines that there is an abnormality when the difference does not become equal to or less than the threshold value even after a predetermined period of time has elapsed since start of heating of the sensitive membrane.

6. The detection device according to claim 2, wherein the arithmetic unit corrects the second detection value based on the first detection value and the first reference value and calculates the determination information based on the second detection value that has been corrected, when the difference does not become equal to or less than the threshold value even after a predetermined period of time has elapsed since start of heating of the sensitive membrane.

7. The detection device according to claim 1, wherein the arithmetic unit acquires a second reference value related to a reference gas detected by the detector after heating of the sensitive membrane is stopped, and calculates the determination information based on the second detection value and the second reference value.

8. The detection device according to claim 1,
wherein the vibrator is provided in a plurality,
wherein the heater is provided in a plurality to heat sensitive membranes of the vibrators, respectively, and
wherein the arithmetic unit calculates the determination information based on the second detection value corresponding to each of the vibrators.

9. The detection device according to claim 8, wherein the control unit heats the sensitive membrane of at least one of the vibrators to a temperature different from that of the sensitive membrane of another vibrator.
